(19) United States
(12) Patent Application Publication
(10) Pub. No.: US 2021/0032684 A1
(43) Pub. Date: Feb. 4, 2021

(54) STABILIZATION OF NON-DENATURED POLYPEPTIDES, NUCLEIC ACIDS, AND EXOSOMES IN A BLOOD SAMPLE AT AMBIENT TEMPERATURES

(71) Applicant: Biomatrica, Inc., San Diego, CA (US)

(72) Inventors: Rolf MULLER, Del Mar, CA (US); Scott WHITNEY, San Diego, CA (US); Paul DIAZ, Riverside, CA (US)

(21) Appl. No.: 17/073,580

(22) Filed: Oct. 19, 2020

Related U.S. Application Data
(63) Continuation of application No. 15/316,680, filed on Dec. 6, 2016.
(60) Provisional application No. 62/010,164, filed on Jun. 10, 2014.

(51) Int. Cl. C12Q 1/6806; C12N 5/078; C12N 15/10; A01N 1/02

(52) U.S. Cl. CPC C12Q 1/6806; A01N 1/0226; C12N 15/10; C12N 5/0634

(57) ABSTRACT

Provided herein are formulations for the stabilization of one or more polypeptide or nucleic acid molecule in a native, non-denatured state at ambient temperatures. Also provided are compositions, articles of manufacture, kits and methods for substantially stable storage of one or more polypeptide or nucleic acid molecule in a native, non-denatured, and/or functionally active conformation substantially free on intracellular polypeptides and nucleic acids at ambient temperatures are provided. Also provided are formulations for the stabilization of one or more exosome at ambient temperatures, and compositions, articles of manufacture, kits and methods of use.

### CROSS-REFERENCE

This application claims the benefit of U.S. Provisional Application No. 62/010,164, filed Jun. 10, 2014, which is incorporated by reference herein in its entirety.

### BACKGROUND OF THE INVENTION

### 1. Technical Field

The present invention relates generally to stabilization of one or more non-denatured nucleic acid molecule and/or polypeptide, or exosome in a blood sample at ambient temperatures. In particular, the invention relates to formulations, compositions, articles of manufacture, kits and methods for substantially stable storage of one or more non-denatured nucleic acid molecule and/or polypeptide, or exosome in a blood sample at ambient temperatures.

### 2. Background

Whole blood is a complex mixture of cells, nucleic acids, proteins and various other analytes. In particular, blood components include, but are not limited to: cells, such as leukocytes (monocytes, lymphocytes and granulocytes), erythrocytes, thrombocytes and circulating tumor cells; nucleic acid molecules, such a circulating-free DNA (cfDNA); polypeptides, such as lipoproteins, albumin and serum proteins, and other various analytes.

A constant need for blood donors exists for whole blood, in part, due to the relatively short half-life and storage requirements for whole blood. For instance, whole blood collected for transfusion must be shipped on ice and stored under cold refrigeration conditions with constant rocking in order to maintain intact, viable cells and to preserve cellular and acellular polypeptides, nucleic acid molecules and other analytes from chemical and enzymatic degradation. This requirement for cold storage conditions can limit the availability of whole blood supplies in areas lacking required cold storage facilities, e.g., due to the lack of sufficient equipment or necessary constant electrical power to maintain adequate cold storage temperatures.

Compositions and methods for stabilizing, shipping and storing whole blood and blood components at ambient temperatures have been investigated. While currently used compositions are capable of preventing degradation of polypeptides and nucleic acid molecules, a major limitation is that the stabilized polypeptides and nucleic acid molecules are maintained in a denatured conformation, e.g., single-stranded DNA and unfolded proteins lacking proper secondary and/or tertiary structure, due to the reactive components used in the storage formulations and compositions. A further limitation is that these formulations can result in the lysis of blood cells thereby releasing the cellular contents into the whole blood sample that can make subsequent quantitation and diagnostic analyses of freely circulating polypeptides and nucleic acid molecules complicated due to contaminating genomic DNA and intracellular proteins.

Non-invasive diagnostic tests have been recently developed to determine abnormalities of fetuses using the circulating cell-free DNA in maternal blood (e.g., MATERNIT21® PLUS non-invasive fetal abnormality test, Sequenom, Inc). The impact of this tool has spurned further research in using the cell-free DNA and cell-free amino acid profiles to predict disease states and the relative progression of certain diseases. The use of the collected blood specimens within a short pre-described time is paramount as changes to the profile occur in a relatively short time at ambient temperatures, and exposure to 4° C. can cause spurious changes in diagnostic markers on cell surfaces and increase the amounts released into the plasma or serum fractions. While collection tubes for stabilizing cell-free nucleic acids for up to 7 days at room temperatures exist (Streck Labs), the main constituents of the formulations provided in the tubes are formaldehyde releasing agents (such as imidazolinylurea or diazolidinyl urea) that react to fix the cells and make them less permeable to agents entering or exiting the cells.

Thus, there is a need to develop new formulations, compositions and methods for the stabilization of nucleic acid molecules and polypeptides, individually or together, in which the substantially stable nucleic acid molecules and polypeptides are maintained in the blood sample in their native, non-denatured conformation at ambient temperatures. The formulations, compositions and methods of the present invention advantageously overcome the aforementioned limitations by maintaining the integrity of blood cells to prevent contamination of circulating nucleic acid molecules and polypeptides by lysed cellular components while also preserving functionally-active polypeptides and nucleic acid molecules in their native conformation at ambient temperatures. These stabilized nucleic acid molecules and polypeptides may be shipped and stored without the need for refrigeration or freezing, and are stable at ambient temperatures for extended periods of time, e.g., days, weeks, months or even years, facilitating the quantitation, analysis and/or use of various nucleic acid molecules and polypeptides for diagnostic and potential therapeutic applications.

### BRIEF SUMMARY OF THE INVENTION

In one aspect of the invention, there are provided formulations for substantially stable storage of one or more polypeptide in a native, non-denatured conformation in a blood sample at ambient temperatures, wherein the one or more polypeptide is stabilized in a native, non-denatured conformation substantially free of contaminating intracellular polypeptides after storage at ambient temperature for a period of at least 3 days. In some embodiments, at least 80% of the polypeptides are stabilized in a native, non-denatured conformation after storage at ambient temperature for a period of at least three days. In some embodiments, the one or more polypeptide remains in a native, non-denatured conformation after storage at ambient temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the formulation comprises: a pH buffer; a non-reducing sugar; a trisaccharide; and one or more of a water-soluble polymer; cationic compound; zwitterionic compound; a phosphatase inhibitor, or a combination thereof. In some embodiments, the non-reducing sugar is sucrose. In some embodiments, the trisaccharide is selected from the group consisting of maltotriose, isomaltotriose, raffinose, melezitose, nigerotriose, and combinations thereof. In some embodiments, the trisaccharide is melezitose. In some embodiments, the water-soluble polymer is polyvinyl alcohol. In some embodiments, the phosphatase inhibitor is 2-glycerol phosphate. In some embodiments, the zwitterionic compound is a compound of formula (I):

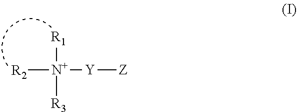

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), wherein A is an unsubstituted or substituted alkyl, aryl, arylalkyl, or any side chain typically found in one of the 20 naturally occurring amino acids; and Z is CO₂—, SO₃— or OPO₃—. In some embodiments, the cationic compound is selected from the group consisting of:

(a) a compound of formula (II):

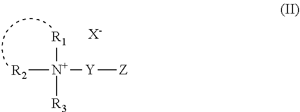

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; Z is CO₂A; and X is a pharmaceutically acceptable anion;

(b) a compound of formula (III):

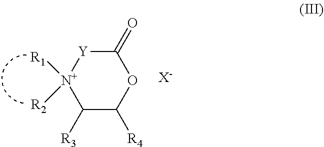

wherein R, R2, R3, and R4 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; and X is a pharmaceutically acceptable anion; and

(c) a compound of formula (IV):

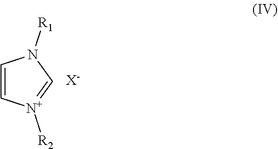

wherein R1 and R2 are independently selected from unsubstituted or substituted alkyl, unsubstituted or substituted aryl, unsubstituted or substituted arylalkyl; and X is a pharmaceutically acceptable anion. In some embodiments, R1 and R2 of a compound of formula (I), formula (II), or formula (III) form a morpholino ring, pyrrolidinium ring, a piperidinium ring, or an oxazinium ring. In some embodiments, the zwitterionic compound is selected from the zwitterionic compounds set forth in Table 1. In some embodiments, the cationic compound is selected from the cationic compounds set forth in Table 1. In some embodiments, the zwitterionic compound is N,N-dimethyl-N-(2-hydroxyethyl)-3-ammonium-proprionate or N-ethyl-piperidinium-4-butylsulfonate. In some embodiments, the one or more polypeptide is selected from the group consisting of an antibody, an enzyme, a plasma protein, a serum protein, and combinations thereof. In some embodiments, the composition comprises: a pH buffer; a non-ionic starch; and one or more of a polyol; a phosphatase inhibitor; an amino acid; or a combination thereof. In some embodiments, the polyol is selected from the group consisting of glycol, glycerol, erythritol, threitol, arabitol, xylitol, ribitol, adonitol, mannitol, sorbitol, galactitol, fucitol, iditol, inositol, and combinations thereof. In some embodiments, the polyol is glycerol. In some embodiments, the phosphatase inhibitor is 2-glycerol phosphate. In some embodiments, the amino acid is glycine. In some embodiments, the amino acid is sarcosine. In some embodiments, the non-reducing sugar is sucrose and the trisaccharide is selected from the group consisting of maltotriose, isomaltotriose, raffinose, melezitose, and nigerotriose, more preferably melezitose. In some embodiments, the water-soluble polymer is polyvinyl alcohol, the phosphatase inhibitor is 2-glycerol phosphate, and the zwitterionic compound is a quaternary inner salt. In some embodiments, the water-soluble polymer is polyvinyl alcohol, the phosphatase inhibitor is 2-glycerol phosphate, and the zwitterionic compound is N,N-dimethyl-N-(2-hydroxyethyl)-3-ammonium-proprionate or N-ethyl-piperidinium-4-butylsulfonate. In certain embodiments, the polyol is selected from the group consisting of glycol, glycerol, erythritol, threitol, arabitol, xylitol, ribitol, adonitol, mannitol, sorbitol, galactitol, fucitol, iditol and inositol, the phosphatase inhibitor is 2-glycerol phosphate, and the amino acid is glycine or sarcosine. In certain embodiments, the polyol is glycerol, the phosphatase inhibitor is 2-glycerol phosphate, and the amino acid is glycine or sarcosine. In some embodiments, the formulation is selected from those set forth in Table 2. In some embodiments, there are provided compositions of a substantially, stably stored one or more purified, non-denatured polypeptide comprising one or more purified, non-denatured polypeptide admixed with any of the foregoing formulations.

Described herein, in some embodiments, are methods for substantially stable storage of one or more polypeptide in a native, non-denatured conformation in a blood sample at ambient temperatures, comprising: admixing a sample of collected blood from a subject with a formulation for substantially stable storage of one or more polypeptide in a native, non-denatured conformation in a blood sample provided herein, wherein the one or more polypeptide remains in a native, non-denatured state after storage at room temperature for a period of at least three days. In some embodiments, the polypeptide is selected from the group consisting of an antibody, an enzyme, a plasma protein, a serum protein, and a combination thereof. In some embodiments, at least 80% of the polypeptides remain in a native, non-denatured state after storage at room temperature for a period of at least three days. In some embodiments, the subject is an animal. In some embodiments, the subject is a mammal. In some embodiments, the subject is a human.

In another aspect of the invention, there are provided formulations for substantially stable storage of one or more nucleic acid molecule in a native, non-denatured state in a blood sample at ambient temperatures, wherein the one or more nucleic acid molecule is stabilized in a native, non-denatured state substantially free of contaminating intracellular nucleic acids after storage at ambient temperature for a period of at least 3 days. In some embodiments, at least 80% of the nucleic acid molecules are stabilized in a native, non-denatured state substantially free of contaminating intracellular nucleic acids after storage at room temperature for a period of at least 3 days. In some embodiments, the one or more nucleic acid molecule remains in a native, non-denatured conformation after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the formulation comprises: a pH buffer; a phosphatase inhibitor; a purine: a zwitterionic compound; a cationic compound; or a combination thereof; and an apoptosis inhibitor or a caspase inhibitor. In some embodiments, the phosphatase inhibitor is 2-glycerol phosphate. In some embodiments, the zwitterionic compound is a quaternary inner salt. In some embodiments, the quaternary inner salt is 2-(benzyl(2-hydroxyethyl)(methyl)ammonio)acetate. In some embodiments, the zwitterionic compound is a compound of formula (I):

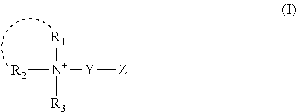

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), wherein A is an unsubstituted or substituted alkyl, aryl, arylalkyl, or any side chain typically found in one of the 20 naturally occurring amino acids; and Z is CO₂—, SO₃— or OPO₃—. In some embodiments, the cationic compound is selected from the group consisting of:

(a) a compound of formula (II):

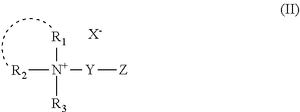

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; Z is CO₂A; and X is a pharmaceutically acceptable anion;

(b) a compound of formula (III):

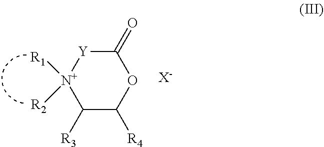

wherein R1, R2, R3, and R4 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; and X is a pharmaceutically acceptable anion; and

(c) a compound of formula (IV):

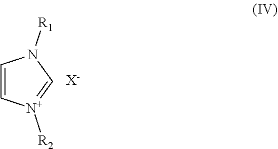

wherein R1 and R2 are independently selected from unsubstituted or substituted alkyl, unsubstituted or substituted aryl, unsubstituted or substituted arylalkyl; and X is a pharmaceutically acceptable anion. In some embodiments, R1 and R2 of a compound of formula (I), formula (II), or formula (III) form a morpholino ring, pyrrolidinium ring, a piperidinium ring, or an oxazinium ring. In some embodiments, the zwitterionic compound is selected from the zwitterionic compounds set forth in Table 1. In some embodiments, the cationic compound is selected from the cationic compounds set forth in Table 1. In some embodiments, the formulation further comprises a nuclease inhibitor. In some embodiments, the purine is adenine. In some embodiments, the nuclease inhibitor is aurin tricarboxylic acid. In some embodiments, the composition further comprises an agent selected from the group consisting of an antibiotic, a purine derivative, and a combination thereof. In some embodiments, the antibiotic is selected from the group consisting of rifampicin, actinomycin D, 5-hydroxy-1,4-napthoquinone, and a combination thereof. In some embodiments, the purine derivative is 5-mercaptopurine. In some embodiments, the caspase inhibitor is Q-VD-OPH. In some embodiments, the one or more nucleic acid molecule is a circulating-free DNA molecule. In certain embodiments, the phosphatase inhibitor is 2-glycerol phosphate, the zwitterionic compound is a quaternary inner salt, more preferably 2-(benzyl(2-hydroxyethyl)(methyl)ammonio)acetate, the apoptosis inhibitor is Apoptosis Inhibitor (Calbiochem Cat #178488), the caspase inhibitor is Q-VD-OPH and the pH buffer is MOPS. In certain embodiments, the phosphatase inhibitor is 2-glycerol phosphate, the zwitterionic compound is 2-(benzyl(2-hydroxyethyl)(methyl)ammonio) acetate, the apoptosis inhibitor is Apoptosis Inhibitor (Calbiochem Cat #178488), the caspase inhibitor is Q-VD-OPH and the pH buffer is MOPS. In certain other embodiments, the formulations comprise: a pH buffer; a phosphatase inhibitor; a purine; a cationic compound or zwitterionic compound; and a nuclease inhibitor. In certain embodiments, the phosphatase inhibitor is 2-glycerol phosphate, the purine is adenine, the zwitterionic compound is a quaternary inner salt, the nuclease inhibitor is aurin tricarboxylic acid and the pH buffer is MOPS. In certain embodiments, the phosphatase inhibitor is 2-glycerol phosphate, the purine is adenine, the zwitterionic compound is 2-(benzyl(2-hydroxyethyl)(methyl)ammonio)acetate, the nuclease inhibitor is aurin tricarboxylic acid and the pH buffer is MOPS. In some embodiments, the formulation further comprises a polar solvent and a chelating agent. In some embodiments, the formulation further comprises dimethylacetamide and tripotassium EDTA. In some embodiments, the formulation is selected from the formulations set forth in Table 3. In some embodiments, there are provided compositions of a substantially, stably stored one or more purified, non-denatured nucleic acid molecule comprising one or more purified, non-denatured nucleic acid molecule admixed with any of the foregoing formulations.

Described herein, in some embodiments, are methods for substantially stable storage of one or more nucleic acid molecule in a native, non-denatured state in a blood sample at ambient temperatures, comprising: admixing a sample of collected blood from a subject with a formulation for substantially stable storage of one or more nucleic acid molecule in a native, non-denatured state in a blood sample at ambient temperatures provided herein, wherein the one or more nucleic acid molecule remains in a native, non-denatured state substantially free of contaminating intracellular nucleic acids after storage at room temperature for a period of at least three days. In some embodiments, the nucleic acid molecule encodes a polypeptide selected from the group consisting of an antibody, an enzyme, and a serum protein an antibody, an enzyme, a plasma protein, and a serum protein. In some embodiments, at least 80% of the nucleic acid molecules remain in a native, non-denatured state substantially free of contaminating intracellular nucleic acids after storage at room temperature for a period of at least three days. In some embodiments, the nucleic acid molecule is a circulating-free DNA molecule. In some embodiments, the subject is an animal. In some embodiments, the subject is a mammal. In some embodiments, the subject is a human.

Described herein, in some embodiments, are formulations for substantially stable storage of one or more exosome in a native state in a blood sample at ambient temperatures, comprising: a pH buffer; a phosphatase inhibitor: a purine: a zwitterionic compound; a cationic compound; or a combination thereof; and an apoptosis inhibitor or a caspase inhibitor, wherein the one or more exosome is stabilized after storage at room temperature for a period of at least three days. In some embodiments, at least 80% of the exosomes are stabilized after storage at room temperature for a period of at least three days. In some embodiments, the one or more exosome is stabilized after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In some embodiments, the phosphatase inhibitor is 2-glycerol phosphate. In some embodiments, the zwitterionic compound is a quaternary inner salt. In some embodiments, the quaternary inner salt is 2-(benzyl(2-hydroxyethyl)(methyl)ammonio)acetate. In some embodiments, the zwitterionic compound is a compound of formula (I):

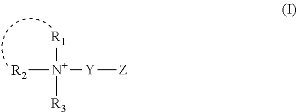

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), wherein A is an unsubstituted or substituted alkyl, aryl, arylalkyl, or any side chain typically found in one of the 20 naturally occurring amino acids; and Z is CO₂—, SO₃— or OPO₃—. In some embodiments, the cationic compound is selected from the group consisting of:

(a) a compound of formula II):

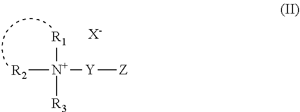

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; Z is CO₂A; and X is a pharmaceutically acceptable anion;

(b) a compound of formula (III):

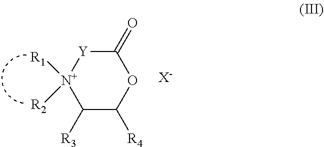

wherein R1, R2, 3, and R4 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; and X is a pharmaceutically acceptable anion; and

(c) a compound of formula (IV):

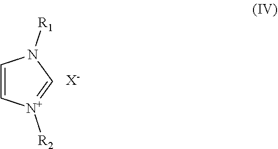

wherein R1 and R2 are independently selected from unsubstituted or substituted alkyl, unsubstituted or substituted aryl, unsubstituted or substituted arylalkyl; and X is a pharmaceutically acceptable anion. In some embodiments, R1 and R2 of a compound of formula (I), formula (II), or formula (III) form a morpholino ring, pyrrolidinium ring, a piperidinium ring, or an oxazinium ring. In some embodiments, the zwitterionic compound is selected from the zwitterionic compounds set forth in Table 1. In some embodiments, the cationic compound is selected from the cationic compounds set forth in Table 1. In some embodiments, the formulation further comprises a nuclease inhibitor. In some embodiments, the purine is adenine. In some embodiments, the nuclease inhibitor is aurin tricarboxylic acid. In some embodiments, the composition further comprises an agent selected from the group consisting of an antibiotic, a purine derivative and a combination thereof. In some embodiments, the antibiotic is selected from the group consisting of rifampicin, actinomycin D, 5-hydroxy-1,4-napthoquinone, and a combination thereof. In some embodiments, the purine derivative is 5-mercaptopurine. In certain embodiments, the phosphatase inhibitor is 2-glycerol phosphate, the zwitterionic compound is a quaternary inner salt, more preferably 2-(benzyl(2-hydroxyethyl)(methyl)ammonio)acetate, the apoptosis inhibitor is Apoptosis Inhibitor (Calbiochem Cat #178488), the caspase inhibitor is Q-VD-OPH and the pH buffer is MOPS. In certain embodiments, the phosphatase inhibitor is 2-glycerol phosphate, the zwitterionic compound is 2-(benzyl(2-hydroxyethyl)(methyl)ammonio)acetate, the apoptosis inhibitor is Apoptosis Inhibitor (Calbiochem Cat #178488), the caspase inhibitor is Q-VD-OPH and the pH buffer is MOPS. In certain embodiments, the formulation comprises a pH buffer, a phosphatase inhibitor, a purine, a cationic compound or zwitterionic compound; and a nuclease inhibitor. In certain embodiments, the phosphatase inhibitor is 2-glycerol phosphate, the purine is adenine, the zwitterionic compound is a quaternary inner salt, the nuclease inhibitor is aurin tricarboxylic acid and the pH buffer is MOPS. In certain embodiments, the phosphatase inhibitor is 2-glycerol phosphate, the purine is adenine, the zwitterionic compound is 2-(benzyl(2-hydroxyethyl)(methyl)ammonio)acetate, the nuclease inhibitor is aurin tricarboxylic acid and the pH buffer is MOPS. In some embodiments, the formulation further comprises a polar solvent and a chelating agent. In some embodiments, the formulation further comprises dimethylacetamide and tripotassium EDTA. In some embodiments, the formulation is selected from the formulations set forth in Table 3. In some embodiments, there are provided compositions of a substantially, stably stored one or more purified exosome comprising one or more purified exosome admixed with any of the foregoing formulations.

Described herein, in some embodiments, are methods for substantially stable storage of one or more exosomes in a native state in a blood sample at ambient temperatures, comprising: admixing a sample of collected blood from a subject with a formulation for substantially stable storage of one or more exosome in a native state in a blood sample at ambient temperatures provided herein, wherein the one or more exosome remains in a native state after storage at room temperature for a period of at least three days. In some embodiments, the subject is an animal. In some embodiments, the subject is a mammal. In some embodiments, the subject is a human.

Described herein, in some embodiments, are articles of manufacture, comprising any one of the formulations described herein, including those set forth in Tables 2 and 3, contained within a blood collection tube. In some embodiments, the blood collection tube is an evacuated blood collection tube.

Described herein, in some embodiments, are kits comprising any one of the articles of manufacture and a package insert.

### DETAILED DESCRIPTION OF THE INVENTION

The present invention relates to formulations, compositions, articles of manufacture, kits and methods for substantially stable storage of one or more polypeptide or nucleic acid molecule in a native, non-denatured conformation in a blood sample at ambient temperatures. The invention also relates, in some embodiments, to formulations, compositions, articles of manufacture, kits and methods for substantially stable storage of one or more exosome in a blood sample at ambient temperatures. In some embodiments, the formulations provide for the substantially stable storage of polypeptides and/or nucleic acid molecules, wherein at least 60%, 70%, 80%, 90%, 95%, or even 99% of the polypeptides and/or nucleic acid molecules remain in a native, non-denatured state after storage at room temperature substantially free of contaminating intracellular components. In some embodiments, the formulations provide for the substantially stable storage of exosomes, wherein at least 60%, 70%, 80%, 90%, 95%, or even 99% of the exosomes are stable after storage at room temperature.

In some embodiments, the formulations provide for substantially stable storage of polypeptides that retain their native conformation and are functionally active. In some embodiments, the compositions described herein also provide substantially stable storage conditions for non-denatured, full-length nucleic acid molecules, such as circulating-free DNA, free of contaminating intracellular DNA for improved quantitation or diagnostic analysis.

### Definitions

Unless defined otherwise, all technical and scientific terms used herein have the same meaning as is commonly understood by one of skill in the art to which this invention belongs. All patents, patent applications and publications referred to herein are incorporated by reference in their entirety.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

“About” as used herein when referring to a measurable value such as an amount, a temporal duration, and the like, is meant to encompass variations of ±20% or ±10%, or ±5%, or even ±1% from the specified value, as such variations are appropriate for the disclosed compositions or to perform the disclosed methods.

As described herein, a nucleic acid molecule refers to a polymer of two or more modified and/or unmodified deoxyribonucleotides or ribonucleotides, either in the form of a separate fragment or as a component of a larger construction. Nucleic acid molecule(s), oligonucleotide(s), and polynucleotide(s), include RNA or DNA (either single or double stranded, coding, complementary or antisense), or RNA/DNA hybrid sequences of more than one nucleotide in either single chain or duplex form, DNA analogs such as PNA (peptide nucleic acid), and any chemical modifications thereof. The DNA may be a single- or double-stranded DNA, cDNA, or a DNA amplified by any amplification technique, or any DNA polymer. In some embodiments, the nucleic acid molecule is a circulating-free DNA (cfDNA). As used herein, cfDNA refers to DNA that is circulating in a subject's blood and not contained within a cell. The RNA may be mRNA, rRNA, tRNA, siRNA, total RNA, small nuclear RNA (snRNA), RNAi, micro RNA, genomic RNA, RNA isolated from cells or tissues, a ribozyme, or any RNA polymer. Encompassed in some embodiments are native nucleic acid molecules, such as those that can be isolated from natural sources. In some embodiments, the nucleic acid molecules are forms, fragments and derivatives derived from natural sources, as well as recombinant forms and artificial molecules, as long as at least one property of the native molecules is present. In some embodiments, the nucleic acid molecules are within biological samples are those that can be applied to analytical, diagnostic and/or pharmaceutical purposes, such as, but not limited to, nucleic acids and their derivatives (e.g. oligonucleotides, DNA, cDNA, PCR products, genomic DNA, plasmids, chromosomes, artificial chromosomes, gene transfer vectors, RNA, mRNA, tRNA, siRNA, miRNA, hnRNA, ribozymes, genomic RNA, peptide nucleic acid (PNA), and bacterial artificial chromosomes (BACs)). In some embodiments, the biological sample is blood.

The term “purified,” as used in “purified polypeptide” or “purified nucleic acid molecule” or “purified exosome” refers to recovery of a nucleic acid molecule, a polypeptide, or exosome, respectively, which is at least 50%, at least 55%, at least 60%, at least 65%, at least 70%, at least 75%, at least 80%, at least 85%, at least 90%, at least 92%, at least 94%, at least 95%, at least 96%, at least 98%, % or at least 99% purified with respect to removal of a contaminant, e.g., cellular components such as protein, lipid or salt. The term “substantially purified” generally refers to separation of a majority of cellular proteins or reaction contaminants from the blood sample, so that compounds capable of interfering with the subsequent use of the isolated biomolecule (such as a nucleic acid molecule) are removed.

As described herein, the term “polypeptide” refers to any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric. The term “polypeptide” encompasses fragments and variants (including fragments of variants) thereof, unless otherwise contradicted by context.

The term “isolated protein” or “isolated polypeptide” is a protein or polypeptide that by virtue of its origin (e.g., a cell or biological sample) or source of derivation is not associated with naturally associated components that accompany it in its native state. Thus, a polypeptide that is chemically synthesized will be “isolated” from its naturally associated components. A protein may also be purified or substantially purified by rendering it substantially free of naturally associated components by isolation at least 50%, at least 55%, at least 60%, at least 65%, at least 70%, at least 75%, at least 80%, at least 85%, at least 90%, at least 92%, at least 94%, at least 95%, at least 96%, at least 98%, % or at least 99% purified with respect to removal of a contaminant, e.g., cellular components such as nucleic acids, lipid or salt using protein purification techniques well-known in the art.

### Formulations and Compositions for Stabilizing Polypeptides, Nucleic Acids or Exosomes in a Native Non-Denatured State in a Blood Sample at Ambient Temperatures

In one aspect of the present invention, formulations and compositions are provided for substantially stable storage of one or more polypeptide in a non-denatured conformation in a blood sample at ambient temperature, wherein the one or more polypeptide is stabilized in a native non-denatured conformation after storage at room temperature for a period of at least three days. In some embodiments, the formulations and compositions are provided for substantially stable of polypeptides in a native, non-denatured conformation in a blood sample at ambient temperatures, wherein at least 80% of the polypeptides are stabilized in a native, non-denatured conformation after storage at room temperature for a period of at least three days. In some embodiments, the one or more polypeptide remains in a native, non-denatured conformation after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

The term “ambient temperature” as used herein refers to common indoor room temperatures. In some embodiments, ambient temperature is 15 to 32° C. In some embodiments, ambient temperature is 20 to 27° C.

In certain embodiments, the formulations for substantially stable storage of one or more polypeptide and/or nucleic acid molecule in a native, non-denatured state in a blood sample at ambient temperatures comprise: a pH buffer: a non-reducing sugar; a trisaccharide; and at least one of a water-soluble polymer, a cationic compound, a zwitterionic compound and a phosphatase inhibitor. In certain embodiments, formulations for substantially stable storage of one or more polypeptide and/or nucleic acid molecule in a native, non-denatured state in a blood sample at ambient temperatures, comprising pH buffer; a non-ionic starch; and one or more of a polyol, a phosphatase inhibitor, and an amino acid, wherein the one or more polypeptide and/or nucleic acid molecule is stabilized in a native, non-denatured state after storage at room temperature for a period of at least three days are provided. In certain embodiments, the polyol is selected from the group consisting of glycol, glycerol, erythritol, threitol, arabitol, xylitol, ribitol, adonitol, mannitol, sorbitol, galactitol, fucitol, iditol and inositol, the phosphatase inhibitor is 2-glycerol phosphate, and the amino acid is glycine or sarcosine. In certain embodiments, the polyol is glycerol, the phosphatase inhibitor is 2-glycerol phosphate, and the amino acid is glycine or sarcosine.

In some embodiments, formulations are provided for substantially stable storage of one or more nucleic acid molecule in a native, non-denatured state in a blood sample at ambient temperatures, wherein the one or more nucleic acid molecule remains in a native, non-denatured state substantially free of contaminating intracellular nucleic acids after storage at room temperature for a period of at least three days. In some embodiments, formulations for substantially stable storage of nucleic acid molecules in a native, non-denatured state in a blood sample at ambient temperatures, wherein at least 80% of the nucleic acid molecules are stabilized in a native, non-denatured state after storage at room temperature for a period of at least three days are provided. In other embodiments, the one or more nucleic acid molecule remains in a native, non-denatured conformation after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

In certain embodiments, the formulations comprise: a pH buffer: a phosphatase inhibitor; a purine: a zwitterionic compound, a cationic compound, or a combination thereof; and an apoptosis inhibitor or a caspase inhibitor. In certain preferred embodiments, the phosphatase inhibitor is 2-glycerol phosphate, the zwitterionic compound is a quaternary inner salt, more preferably 2-(benzyl(2-hydroxyethyl)(methyl)ammonio)acetate, the apoptosis inhibitor is Apoptosis Inhibitor (Calbiochem Cat #178488), the caspase inhibitor is Q-VD-OPH and the pH buffer is MOPS.

In certain other embodiments, the formulations for substantially stable storage of one or more nucleic acid in a native, non-denatured state in a blood sample at ambient temperature comprise: a pH buffer; a phosphatase inhibitor; a purine; a zwitterionic compound, acationic compound, or a combination thereof; and a nuclease inhibitor. In certain embodiments, the phosphatase inhibitor is 2-glycerol phosphate, the purine is adenine, the zwitterionic compound is a quaternary inner salt, more preferably 2-(benzyl(2-hydroxyethyl)(methyl)ammonio)acetate, the nuclease inhibitor is aurin tricarboxylic acid and the pH buffer is MOPS. In some embodiments, the formulations further comprise one of the following: an antibiotic, a purine derivative, an apoptosis inhibitor, a caspase inhibitor, or a combination thereof. In some embodiments, the formulation comprises a polar solvent and a chelating agent. In some embodiments, the formulation further comprises dimethylacetamide and tripotassium EDTA.

In some embodiments, compositions are provided herein in which the blood sample is admixed with the nucleic acid, polypeptide, or exosome and the stabilization formulation to produce substantially stable one or more non-denatured nucleic acid, polypeptide, or exosome, respectively in a whole blood preparation. In some embodiments, a composition comprising purified or substantially purified one or more nucleic acid molecule, polypeptide, or exosome admixed with a corresponding stabilization formulation are provided.

### Formulation Reagents

A. pH Buffers

According to certain embodiments, the herein described formulations and compositions for substantially stable storage of a nucleic acid molecule, polypeptide, or exosome in a blood sample at ambient temperatures include one or more pH buffers. In some embodiments, the buffer is any of a large number of compounds known in the art for their ability to resist changes in the pH of a solution, such as an aqueous solution, in which the pH buffer is present. Selection of one or more particular pH buffers for inclusion in a stable storage composition may be done based on the present disclosure and according to routine practices in the art, and may be influenced by a variety of factors including the pH that is desirably to be maintained, the nature of the sample to be stabilized, the solvent conditions to be employed, the other components of the formulation to be used, and other criteria. For example, typically a pH buffer is employed at a pH that is within about 0.1, 0.2, 0.3, 0.4, 0.5, 0.6, 0.7, 0.8, 0.9 or 1.0 pH unit of a proton dissociation constant (pKₐ) that is a characteristic of the buffer.

Non-limiting examples of pH buffers include citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-1-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N-[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane) and bis-tris (2-[bis(2-hydroxyethyl)amino]-2-(hydroxymethyl)-1,3-propanediol). Certain embodiments contemplated herein, including a number of those set forth in Table 2 or Table 3, may feature a formulation having a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0.

B. Cationic Compounds and Zwitterionic Compounds

In certain embodiments described herein, the zwitterionic compound is a quaternary inner salt. In some embodiments, the quaternary inner salts for substantially stable storage of nucleic acids are those disclosed in WO 2012/018638.

In some embodiments, the zwitterionic compound is a compound of formula (I):

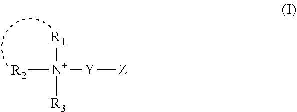

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), wherein A is an unsubstituted or substituted alkyl, aryl, arylalkyl, or any side chain typically found in one of the 20 naturally occurring amino acids; and Z is CO₂—, SO₃— or OPO₃—. In some embodiments, R1 and R2 form a morpholino ring, pyrrolidinium ring, a piperidinium ring, or an oxazinium ring.

In some embodiments, the cationic compound is selected from the group consisting of:

(a) a compound of formula (II):

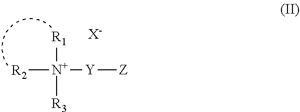

wherein R1, R2, and R3 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; Z is CO₂A; and X is a pharmaceutically acceptable anion;

(b) a compound of formula (III):

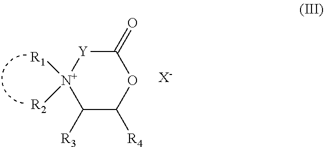

wherein R1, R2, R3, and R4 are independently selected from unsubstituted or substituted alkyl, unsubstituted or substituted aryl, unsubstituted or substituted arylalkyl, or R1 and R2 optionally form a ring, Y is CH₂, CH(A), CH(A)-CH(A), CH(A)-CH(A)-CH(A), where A is an unsubstituted or substituted alkyl, aryl, arylalkyl or any side chain typically found in one of the 20 naturally occurring amino acids; and X is a pharmaceutically acceptable anion; and

(c) a compound of formula (IV):

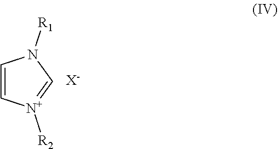

wherein R1 and R2 are independently selected from unsubstituted or substituted alkyl, unsubstituted or substituted aryl, unsubstituted or substituted arylalkyl; and X is a pharmaceutically acceptable anion. In some embodiments, R1 and R2 of a compound of formula (II) or formula (III) form a morpholino ring, pyrrolidinium ring, a piperidinium ring, or an oxazinium ring.

An “alkyl” group refers to an aliphatic hydrocarbon group. The alkyl moiety includes a “saturated alkyl” group, which means that it does not contain any alkene or alkyne moieties. The alkyl moiety also includes an “unsaturated alkyl” moiety, which means that it contains at least one alkene or alkyne moiety. An “alkene” moiety refers to a group that has at least one carbon-carbon double bond, and an “alkyne” moiety refers to a group that has at least one carbon-carbon triple bond. The alkyl moiety, whether saturated or unsaturated, includes branched, straight chain, or cyclic moieties. Depending on the structure, an alkyl group includes a monoradical or a diradical (i.e., an alkylene group), and if a “lower alkyl” having 1 to 6 carbon atoms. As used herein, C1-Cx includes C1-C2, C1-C3 . . . C1-Cx. The “alkyl” moiety optionally has 1 to 10 carbon atoms (whenever it appears herein, a numerical range such as “1 to 10” refers to each integer in the given range; e.g., “1 to 10 carbon atoms” means that the alkyl group is selected from a moiety having 1 carbon atom, 2 carbon atoms, 3 carbon atoms, etc., up to and including 10 carbon atoms, although the present definition also covers the occurrence of the term “alkyl” where no numerical range is designated). The alkyl group of the compounds described herein may be designated as “C1-C4 alkyl” or similar designations. By way of example only, “C1-C4 alkyl” indicates that there are one to four carbon atoms in the alkyl chain, i.e., the alkyl chain is selected from among methyl, ethyl, propyl, iso-propyl, n-butyl, iso-butyl, sec-butyl, and t-butyl. Thus C1-C4 alkyl includes C1-C2 alkyl and C1-C3 alkyl. Alkyl groups are optionally substituted or unsubstituted. Typical alkyl groups include, but are in no way limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, cyclohexyl, and the like.

As used herein, the term “ring” refers to any covalently closed structure. Rings include, for example, carbocycles (e.g., aryls and cycloalkyls), heterocycles (e.g., heteroaryls and non-aromatic heterocycles), aromatics (e.g. aryls and heteroaryls), and non-aromatics (e.g., cycloalkyls and non-aromatic heterocycles). Rings can be optionally substituted. Rings can be monocyclic or polycyclic.

The term “aryl” used alone or as part of a larger moiety as in “arylalkyl” “arylalkoxy”, or “aryloxyalkyl”, refers to a monocyclic, bicyclic or tricyclic, carbon ring system, that includes fused rings, wherein at least one ring in the system is aromatic. The term “aryl” may be used interchangeably with the term “aryl ring”. In one embodiment, aryl includes groups having 6-12 carbon atoms. In another embodiment, aryl includes groups having 6-10 carbon atoms. Examples of aryl groups include phenyl, naphthyl, anthracyl, phenanthrenyl, naphthacenyl, 1,2,3,4-tetrahydronaphthalenyl, 1H-indenyl, 2,3-dihydro-H-indenyl, and the like. A particular aryl is phenyl. In another embodiment aryl includes indanyl, naphthyl, and tetrahydronaphthyl, and the like, where the radical or point of attachment is on an aromatic ring.

The term “optionally substituted” or “substituted” means that the referenced group may be substituted with one or more additional group(s) individually and independently selected from alkyl, cycloalkyl, aryl, heteroaryl, heteroalicyclic, hydroxy, alkoxy, aryloxy, alkylthio, arylthio, alkylsulfoxide, arylsulfoxide, alkylsulfone, arylsulfone, cyano, halo, acyl, nitro, haloalkyl, fluoroalkyl, amino, including mono and di substituted amino groups, and the protected derivatives thereof. By way of example an optional substituents may be LsRs, wherein each Ls is independently selected from a bond, —O—, —C(═O)—, —S—, —S(═O)—, —S(═O)₂—, —NH—, —NHC(O)—, —C(O)NH—, S(═O)₂NH—, —NHS(═O)₂, —OC(O)NH—, —NHC(O)O—, -(substituted or unsubstituted C1-C6 alkyl), or -(substituted or unsubstituted C2-C6 alkenyl); and each Rs is independently selected from H, substituted or unsubstituted C1-C4 alkyl, substituted or unsubstituted C3-C6 cycloalkyl, heteroaryl, or heteroalkyl.

In certain other embodiments of the formulations described herein, including those set forth in Table 2 or Table 3, the cationic compound or zwitterionic compound is selected from one of the exemplary compounds of Table 1.

TABLE 1
Exemplary Cationic and Zwitterionic Compounds

| | | |
|---|---|---|
| D201 | 4-(2-ethoxy-2-oxoethyl)-4-ethylmorpholin- | Cationic |
| | 4-ium bromide | Compound |
| D202 | N-(2-ethoxy-2-oxoethyl)-3-hydroxy-N,N-bis | Cationic |
| | (2-hydroxyethyl)propan-1-aminium bromide | Compound |
| D203 | 2-ethoxy-N,N,N-triethyl-2-oxoethanaminium | Cationic |
| | bromide | Compound |
| D204 | 2-((3-hydroxypropyl)dimethylammonio)acetate | Zwitterion |
| D205 | 2-((2-hydroxypropyl)dimethylammonio)acetate | Zwitterion |
| D206 | 2-(2-(hydroxymethyl)-1-methylpiperidinium- | Zwitterion |
| | 1-yl)acetate | |
| D207 | 2-((2-hydroxyethyl)dimethylammonio)acetate | Zwitterion |
| D208 | 2-((2,3-dihydroxypropyl) dimethylammonio) | Zwitterion |
| | acetate | |
| D209 | 1-(2-ethoxy-2-oxoethyl)-4-hydroxy-1- | Cationic |
| | methylpiperidinium bromide | Compound |
| D210 | 2-(4-hydroxy-1-methylpiperidinium-1-yl) | Zwitterion |
| | acetate | |
| D211 | 2-ethoxy-N-(2-(2-hydroxyethoxy)ethyl)- | Cationic |
| | N,N-dimethyl-2-oxoethanaminium bromide | Compound |
| D212 | 2-((2-(2-hydroxyethoxy)ethyl)dimethylammonio) | Zwitterion |
| | acetate | |
| D213 | 4-(2-hydroxyethyl)-4-methyl-2-oxomorpholin- | Cationic |
| | 4-ium bromide | Compound |
| D214 | 2-(bis(2-hydroxyethyl)-(methyl)ammonio)acetate | Zwitterion |
| D215 | 2-(4-(2-hydroxyethyl)morpholino-4-ium)acetate | Zwitterion |
| D216 | 2-(4-(2-hydroxyethyl)morpholino-4-ium)acetate | Zwitterion |
| D217 | 4-(2-ethoxy-2-oxoethyl)-4-methylmorpholin- | Cationic |
| | 4-ium bromide | Compound |
| D218 | 1-(2-ethoxy-2-oxoethyl)-1-methylpyrrolidinium | Cationic |
| | bromide | Compound |
| D219 | 2-(benzyl(2-hydroxy-ethyl)(methyl)ammonio) | Zwitterion |
| | acetate | |
| D220 | 3-(2,3-dihydroxypropyl)-1-methyl-1H-imidazol- | Cationic |
| | 3-ium chloride | Compound |
| D221 | 1,3-dimethyl-1H-imidazol-3-ium methyl sulfate | Cationic |
| | | Compound |
| D222 | 3-(2-ethoxy-2-oxoethyl)-1-methyl-1H-imidazol- | Cationic |
| | 3-ium bromide | Compound |
| D223 | 2-(1-(2-hydroxyethyl) pyrrolidinium-1-yl) | Zwitterion |
| | acetate | |
| D224 | N-benzyl-2-ethoxy-N,N-dimethyl-2- | Cationic |
| | oxoethanaminium bromide | Compound |
| D225 | 2-ethoxy-N,N-diethyl-N-methyl-2- | Cationic |
| | oxoethanaminium bromide | Compound |
| D226 | N-(2-ethoxy-2-oxoethyl)-N,N-dimethylbutan- | Cationic |
| | 1-aminium bromide | Compound |
| D227 | 1-(2-ethoxy-2-oxoethyl)-1-methylpiperidinium | Cationic |
| | bromide | Compound |
| D228 | N-(2-ethoxy-2-oxoethyl)-N,N- | Cationic |
| | dimethylbenzenaminium bromide | Compound |
| D229 | 1-(2-ethoxy-2-oxoethyl)-3-hydroxy-1- | Cationic |
| | methylpiperidinium bromide | Compound |
| D230 | 3-(2-(2-hydroxyethoxy)ethyl)-1-methyl-1H- | Cationic |
| | imidazol-3-ium chloride | Compound |
| D231 | 3-(2-(2-(2-hydroxyethoxy)ethoxy)ethyl)-1- | Cationic |
| | methyl-1H-imidazol-3-ium chloride | Compound |
| D232 | 1-methyl-3-tetradecyl-1H-imidazol-3-ium | Cationic |
| | bromide | Compound |
| D233 | N-(2-ethoxy-2-oxoethyl)-N,N- | Cationic |
| | dimethylcyclohexanaminium bromide | Compound |
| D234 | 3-((2-hydroxy-ethyl)dimethyl-ammonio) | Zwitterion |
| | propanoate | |

In certain embodiments for substantially stable storage of one or more polypeptide, including those set forth in Table 2, the zwitterionic compound is a quaternary inner salt. In certain embodiments, the quaternary inner salt is N,N-dimethyl-N-(2-hydroxyethyl)-3-ammonium-proprionate or N-ethyl-piperidinium-4-butylsulfonate, at concentrations of about 1.0-100 mg/mL, or 1.0-50 mg/mL, or 10.0-50 mg/mL. In certain embodiments for substantially stable storage of one or more nucleic acid molecule, including those set forth in Table 3, the quaternary inner salt is 2-(benzyl(2-hydroxyethyl)(methyl)ammonio)acetate.

C. Non-Reducing Sugars

Also as described herein, in some embodiments, are formulations that include at least one non-reducing sugar in the composition for substantially stable storage of one or more polypeptide in a native conformation in a blood sample at ambient temperatures. Non-reducing sugars are carbohydrate molecules that lack a functional aldehyde group. Exemplary non-reducing sugars include sucrose and trehalose. In some embodiments the non-reducing sugar is trehalose and is present at a concentration of about 1.0-50 mM, or about 10.0-30 mM, or about 25 mM. In some embodiments for substantially stable storage of one or more polypeptide in a native conformation, the non-reducing sugar is sucrose present at a concentration of about 1.0-50 mM, or about 1.0-30 mM, or about 10 mM.

D. Trisaccharides

As described herein, in certain embodiments, the formulations include at least one trisaccharide in the formulation or composition for substantially stable storage of one or more polypeptide in a native conformation in a whole blood sample at ambient temperatures. Trisaccharides are oligosaccharides composed of three monosaccharide monomers with glycosidic bonds connecting them. The glycosidic bond can be formed between any hydroxyl group on the component monosaccharides and different bond combinations (regiochemistry) and stereochemistry (alpha- or beta-) result in trisaccharides that are diastereoisomers with different chemical and physical properties. Selection of one or more particular trisaccharide for inclusion in a stable storage composition may be done based on the present disclosure and according to routine practices in the art, and may be influenced by a variety of factors including other formulation components Exemplary trisaccharides include, but are not limited to, maltotriose, isomaltotriose, raffinose, melezitose, and nigerotriose. In certain embodiments for stabilizing polypeptides, including formulations set forth in Table 2, the trisaccharide is melezitose. In some embodiments, the melezitose is present at a concentration of about 1%-20%, or about 5.0-15%.

E. Polyols

Also as described herein, certain embodiments include at least one polyol in the composition for substantially stable storage of one or more polypeptide in a native conformation in a whole blood sample at ambient temperatures. Polyols are polyhydric alcohols containing two or more hydroxyl groups and have the general formula H(CHOH)ₙH, wherein n is an integer selected from 2 to 7, inclusive. Polyols differ in chain length with most polyols having five- or six-carbon chains being derived from pentoses (five-carbon sugars) and hexoses (six-carbon sugars); however shorter and longer carbon chain polyols also exist. Exemplary polyols include, but are not limited to, glycol, glycerol, erythritol, threitol, arabitol, xylitol, ribitol, adonitol, mannitol, sorbitol, galactitol, fucitol, iditol and inositol. Selection of one or more particular polyols for inclusion in a substantially stable storage composition may be done based on the present disclosure and according to routine practices in the art, and may be influenced by a variety of factors including other formulation components. In certain embodiments, the polyol present in the formulation, including those in Table 2, is a pentose polyol, and is present at a concentration between 20-100 mM, or about 25-75 mM. In certain embodiments, the polyol present in the formulation, including those in Table 2, is adonitol, and is present at a concentration between 20-100 mM, or about 25-75 mM.

F. Water-Soluble Polymers

As described herein, certain embodiments include at least one water-soluble polymer in the formulations and compositions for substantially stable storage of one or more polypeptide in a native conformation in a whole blood sample at ambient temperatures. Such water-soluble polymers include polyvinyl alcohol. It will be appreciated that from the present disclosure the skilled person may select other water-soluble polymers for use in a stable storage formulations and compositions, as may vary based on the other components of the composition that are employed, the particular biological sample being stored, whether nucleic acid molecules or polypeptide molecules or both are sought to be recovered, and other factors. Certain embodiments, including but not limited to those presented in Table 2, contemplate inclusion of a water-soluble polymer at a concentration (on a volumetric basis, i.e., vol/vol) of about 0.1 to 10% (vol/vol), or about 0.1 to 5% (vol/vol), or 1.0% (vol/vol). In certain embodiments, the water-soluble polymer is polyvinyl alcohol with a molecular weight range of about 30-70,000 daltons and about 87-90% hydrolyzed.

G. Phosphatase Inhibitors

The herein described formulations for substantial stable storage of one or more nucleic acid molecule or polypeptide in a non-denatured state in a blood sample at ambient temperatures, in certain embodiments, contain a phosphatase inhibitor. In certain embodiments, the phosphatase inhibitor is an inhibitor of the serine-threonine class of phosphatases. In certain embodiments, the phosphatase inhibitor is 2-glycerol phosphate. For stabilizing polypeptides, the concentration is about 1.0-100 mM, or about 25-75 mM or about 50 mM; and for nucleic acid molecules it is about 1.0-200 mM, or about 25-150 mM, or about 125 mM; and for exosomes it is about 1.0-200 mM, or about 25-150 mM, or about 125 mM.

H. Nonionic Starches

In certain embodiments, the formulations for substantially stable storage of one or more polypeptide in a non-denatured conformation in a blood sample at ambient temperatures, in certain embodiments, contain a nonionic starch. In certain embodiments, the nonionic starch is hydroxyethyl starch (HES). Hydroxyethyl starch is one of the most frequently used volume expanders under the trade names HESPAN by B. Braun Medical Inc. In certain embodiments, HES is present at a concentration of about 1.0-10%, or about 1.0-5.0%, or about 1.0-2.0%.

I. Chelating Agents

Chelating agents or chelators, according to certain embodiments, are included in the presently described formulations and compositions for substantially stable storage of one or more polypeptide or nucleic acid molecule in a non-denatured state in a blood sample, and are known to those familiar with the art for their ability to complex with and hinder the reactivity of metal cations. Exemplary chelating agents include diethylenetriaminepentaacetic acid (DTPA), ethylenediaminetetraacetic acid (EDTA), ethylene glycol tetraacetic acid (EGTA), trans-1,2-diaminocyclohexane-N,N,N′,N′-tetraacetic acid (CDTA), 1,2-bis(2-aminophenoxy)ethane-N,N,N′,N′-tetraacetic acid (BAPTA), 1,4,7,10-tetraazacyclododecane-1,4,7,10-tetraacetic acid (DOTA), N-(2-hydroxyethyl)ethylenediamine-N,N′,N′-triacetic acid, sodium gluconate, and nitrilotriacetic acid (NTA). In some embodiments, the chelating agent is disodium or tripotassium EDTA and is present at a concentration of about 1.0-100 mM, or about 10-90 mM, or about 70 mM.

J. Nuclease Inhibitors

In certain embodiments, the formulations for substantially stable storage of one or more non-denatured nucleic acid molecule in a blood sample at ambient temperatures contain a nuclease inhibitor. Nuclease inhibitors are well-known to those skilled in the art. In some embodiments, any such nuclease inhibitor is used in the formulations, including those in Table 3, compositions and methods of the present invention. In some embodiments, the nuclease inhibitor is aurin tricarboxylic acid.

K. Purine and Purine Derivatives

A purine or purine derivative, according to certain embodiments, is included in the presently described composition for substantially stable storage of nucleic acid molecules in a blood sample. In some embodiments, the purine is adenine, guanine, or a combination thereof. In some embodiments, the purine is a purine derivative. In some embodiments, the purine derivative is 2-mercaptopurine.

L. Antibiotics

In certain embodiments, the formulations for substantially stable storage of one or more non-denatured nucleic acid molecule in a blood sample at ambient temperatures contain an antibiotic. Antibiotics are well-known to those skilled in the art. In some embodiments, any such antibiotic is used in the formulations, compositions and methods of the present invention. In some embodiments, the antibiotic is rifampicin, actinomycin D, 5-hydroxy-1,4-napthoquinone, or a combination thereof.

M. Polar Solvents

In certain embodiments, the formulations for substantially stable storage of one or more non-denatured nucleic acid molecule in a blood sample at ambient temperatures contain a polar solvent. In some embodiments, the polar solvent is dimethylacetamide. In some embodiments, the polar solvent is present at a concentration of 25%. In some embodiments, the polar solvent is dimethylacetamide, and is present at a concentration of 25%.

### Formulations to Stabilize Non-Denatured, Native Polypeptides, Nucleic Acid Molecules, or Exosomes in a Blood Sample at Ambient Temperatures

Described herein, in some embodiments, are formulations for substantially stable storage of one or more non-denatured polypeptide in a blood sample at ambient temperature comprise: a pH buffer; a non-reducing sugar; a trisaccharide; and at least one of a water-soluble polymer, zwitterionic compound, a cationic compound or a phosphatase inhibitor, wherein the one or more polypeptide and/or nucleic acid molecule remains in a native, non-denatured state after storage at room temperature for a period of at least three days. In other embodiments, the one or more polypeptide or nucleic acid molecule remains in a native, non-denatured conformation substantially free of contaminating intracellular polypeptides after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

In certain embodiments, the disaccharide is sucrose and the trisaccharide is selected from the group consisting of maltotriose, isomaltotriose, raffinose, melezitose, and nigerotriose, more preferably melezitose. In still other preferred embodiments, the water-soluble polymer is polyvinyl alcohol, the phosphatase inhibitor is 2-glycerol phosphate, and the zwitterionic compound is a quaternary amine, preferably N,N-dimethyl-N-(2-hydroxyethyl)-3-ammonium-proprionate or N-ethyl-piperidinium-4-butylsulfonate.

In certain preferred embodiments, the formulations for substantially stable storage of non-denatured polypeptides in a blood sample at ambient temperatures are selected from Table 2.

TABLE 2
Exemplary Formulations for Stabilizing Non-denatured Polypeptides
in a Blood Sample at Ambient Temperatures

| Formulation | Composition |
|---|---|
| A | 10% Sucrose, 6% melezitose 1% polyvinyl alcohol (30-70K mol wt and |
| | 87-90% hydrolyzed), |
| | 50 mM Tris pH 8, 1 mM ZnSO₄, Complete Protease Inhibitor (Roche) |
| B | 10% Sucrose, 6% melezitose 1% polyvinyl alcohol (30-70K mol wt and |
| | 87-90% hydrolyzed), |
| | 50 mM Tris pH 8, 25 mg/mL N,N-dimethyl-N-(2-hydroxyethyl)-3-ammonium-proprionate |
| C | 10% Sucrose, 6% melezitose 1% polyvinyl alcohol (30-70K mol wt and |
| | 87-90% hydrolyzed), |
| | 50 mM Tris pH 8, 25 mg/mL N-ethyl-piperidinium-4-butylsulfonate |
| D | 900 mM 2-Glycerol phosphate disodium salt hydrate, |
| | 1.8% hydroxyethyl starch, 0.9X PBS |
| E | 2 M Glycine, 0.1% 2-hydroxyethyl starch, 2X PBS, pH 7.4 |
| F | 10% Sucrose, 6% melezitose, 0.5 M 2-glycerol phosphate disodium salt |
| | hydrate, 50 mM Tris-HCl, pH 8 |
| G | 0.45 M Sarcosine, 1.35 M glycine, 50 mM Tris-HCl, pH 8 |

In other aspects, formulations are provided for substantially stable storage of one or more nucleic acid molecule in a native, non-denatured state in a blood sample at ambient temperatures, wherein the one or more nucleic acid molecule is stabilized in a native, non-denatured state substantially free of intracellular nucleic acids after storage at room temperature for a period of at least three days. In certain embodiments, the formulations comprise: a pH buffer; a phosphatase inhibitor; a purine; a zwitterionic compound, a cationic compound, or a combination thereof; and an apoptosis inhibitor or a caspase inhibitor. In certain embodiments, the phosphatase inhibitor is 2-glycerol phosphate, the zwitterionic compound is a quaternary inner salt, the apoptosis inhibitor is Apoptosis Inhibitor (Calbiochem Cat #178488), the caspase inhibitor is Q-VD-OPH, and the pH buffer is MOPS. In certain embodiments, the phosphatase inhibitor is 2-glycerol phosphate, the zwitterionic compound is 2-(benzyl(2-hydroxyethyl)(methyl)ammonio) acetate, the apoptosis inhibitor is Apoptosis Inhibitor (Calbiochem Cat #178488), the caspase inhibitor is Q-VD-OPH, and the pH buffer is MOPS. In other embodiments, the one or more nucleic acid molecule remains in a native, non-denatured conformation substantially free of intracellular nucleic acids after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

In certain other embodiments, the formulations comprise: a pH buffer; a phosphatase inhibitor; a purine; a zwitterionic compound, a cationic compound, or a combination thereof; and a nuclease inhibitor. In certain embodiments, the phosphatase inhibitor is 2-glycerol phosphate, the purine is adenine, the zwitterionic compound is a quaternary inner salt, more preferably 2-(benzyl(2-hydroxyethyl)(methyl)ammonio)acetate, the nuclease inhibitor is aurin tricarboxylic acid, and the pH buffer is MOPS. In other embodiments, the formulations may further comprise one of the following: an antibiotic, a purine derivative, an apoptosis inhibitor, or a caspase inhibitor. In some embodiments, the formulation further comprises a polar solvent and a chelating agent. In some embodiments, the formulation comprises dimethylacetamide and tripotassium EDTA.

In certain embodiments, the formulations for substantially stable storage of non-denatured nucleic acid molecules in a blood sample at ambient temperatures are selected from Table 3.

TABLE 3
Exemplary Formulations for Stabilizing Non-denatured Nucleic
Acid Molecules in a Blood Sample at Ambient Temperatures

| Formulation | Composition |
|---|---|
| 1 | 500 mM 2-(Benzyl(2-hydroxyethyl)(methyl)ammonio) |
| | acetate, 125 mM 2-glycerol phosphate disodium |
| | salt, 5 mM adenine, 125 mM MOPS, 4 μM Apoptosis |
| | Inhibitor (Calbiochem Cat #178488), pH 6.8 |
| 2 | 500 mM 2-(Benzyl(2-hydroxyethyl)(methyl)ammonio) |
| | acetate, 125 mM 2-glycerol phosphate disodium |
| | salt, 5 mM adenine, 125 mM MOPS, 4 μM Q-VD-OPH |
| | (caspase inhibitor), pH 6.8 |
| 3 | 2.5 M Dihydroxyacetone, 70 mM disodium EDTA, |
| | 2 μM Q-VD-OPH (caspase inhibitor) |
| 4 | 25% Dimethylacetamide, 70 mM tripotassium EDTA |
| 5 | 25% Dimethylacetamide, 70 mM tripotassium EDTA, |
| | 2 uM Q-VD-OPH (caspase inhibitor) |
| 6 | 500 mM 2-(Benzyl(2-hydroxyethyl)(methyl)ammonio) |
| | acetate, 125 mM 2-glycerol phosphate disodium |
| | salt, 10 mM adenine, 10 μM aurin tricarboxylic |
| | acid, 125 mM MOPS, 10 μM rifampicin, pH 6.8 |
| 7 | 500 mM 2-(Benzyl(2-hydroxyethyl)(methyl)ammonio) |
| | acetate, 125 mM 2-glycerol phosphate disodium |
| | salt, 10 mM adenine, 10 μM aurin tricarboxylic |
| | acid, 125 mM MOPS, 10 μM 6-mercaptopurine |
| | monohydrate, pH 6.8 |
| 8 | 500 mM 2-(Benzyl(2-hydroxyethyl)(methyl)ammonio) |
| | acetate, 125 mM 2-glycerol phosphate disodium |
| | salt, 10 mM adenine, 10 μM aurin tricarboxylic |
| | acid, 125 mM MOPS, 10 μM |
| | 5-hydroxy-1,4-napthoquinone, pH 6.8 |
| 9 | 500 mM 2-(Benzyl(2-hydroxyethyl)(methyl)ammonio) |
| | acetate, 125 mM 2-glycerol phosphate disodium |
| | salt, 10 mM adenine, 10 μM aurin tricarboxylic |
| | acid, 125 mM MOPS, 10 μM Actinomycin D, pH 6.8 |
| 10 | 500 mM 2-(Benzyl(2-hydroxyethyl)(methyl)ammonio) |
| | acetate, 125 mM 2-glycerol phosphate disodium |
| | salt, 10 mM adenine, 10 μM aurin tricarboxylic |
| | acid, 125 mM MOPS, 10 μM Q-VD-OPH (caspase |
| | inhibitor), pH 6.8 |

In other aspects, formulations are provided for substantially stable storage of one or more exosome in a native state in a blood sample at ambient temperatures for a period of at least three days. In certain embodiments, the formulations comprise: a pH buffer: a phosphatase inhibitor; a purine: a zwitterionic compound, a cationic compound, or a combination thereof; and an apoptosis inhibitor or a caspase inhibitor. In certain embodiments, the phosphatase inhibitor is 2-glycerol phosphate, the zwitterionic compound is a quaternary inner salt, the apoptosis inhibitor is Apoptosis Inhibitor (Calbiochem Cat #178488), the caspase inhibitor is Q-VD-OPH, and the pH buffer is MOPS. In certain embodiments, the phosphatase inhibitor is 2-glycerol phosphate, the zwitterionic compound is 2-(benzyl(2-hydroxyethyl)(methyl)ammonio) acetate, the apoptosis inhibitor is Apoptosis Inhibitor (Calbiochem Cat #178488), the caspase inhibitor is Q-VD-OPH, and the pH buffer is MOPS. In other embodiments, the exosome remains in a native state after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days. In certain embodiments, the formulations comprise: a pH buffer; a phosphatase inhibitor; a purine; a zwitterionic compound or cationic compound; and a nuclease inhibitor. In certain embodiments, the phosphatase inhibitor is 2-glycerol phosphate, the purine is adenine, the zwitterionic compound is a quaternary inner salt, more preferably 2-(benzyl(2-hydroxyethyl)(methyl)ammonio)acetate, the nuclease inhibitor is aurin tricarboxylic acid, and the pH buffer is MOPS. In other embodiments, the formulations may further comprise one of the following: an antibiotic and a purine derivative. In some embodiments, the formulation further comprises a polar solvent and a chelating agent. In some embodiments, the formulation comprises dimethylacetamide and tripotassium EDTA.

In certain embodiments, the formulation for substantially stable storage exosomes in a blood sample at ambient temperatures is selected from Table 3.

### Methods for Preparing Formulations for Stabilizing Non-Denatured Nucleic Acid Molecules and Polypeptides at Ambient Temperatures

The present formulations may be prepared using commercially available reagents and methods well-known to those skilled in the art. In some embodiments, the formulations are prepared as concentrated stock solutions of the formulation reagents, e.g., 2×, 5×, 10×, 20× or the like, and admixed with the blood sample at the appropriate concentrations. In some embodiments, the blood sample is admixed with the formulation stock at an equal volume (1:1).

### Purified Nucleic Acid Molecules and Polypeptides

In some embodiments, the substantially stable one or more polypeptide and/or nucleic acid molecule in a blood sample at ambient temperatures is further purified using well-known conventional methods routinely employed by those skilled in the art. Apparatus, kits, and methods for purifying nucleic acid molecules and polypeptides from blood are well-known. For instance, substantially stabilized polypeptides and nucleic acids are purified, in some embodiments, by affinity chromatography, gel electrophoresis, or the like. In some embodiments, the purified one or more polypeptide or nucleic acid molecule is subsequently stored in the formulations described herein for extended periods before analysis.

### Articles of Manufacture

In certain embodiments, articles of manufacture are provided in which one of the herein described formulations, including those set forth in Tables 2 or 3, are contained within a suitable blood collection tube, container or vessel. In some embodiments, these articles of manufacture are used for substantially stable storage of one or more blood component by stabilizing one or more blood component at the time of blood collection. In certain embodiments, the blood collection tube is an evacuated blood tube having less than atmospheric pressure to withdraw a predetermined volume of whole blood. In some embodiments, these articles of manufacture are used in the herein described kits and methods.

### Kits

In certain aspects of the invention, there are provided kits comprising any one of the articles of manufacture comprising the formulations of the present invention and a package insert. In some embodiments, the components of the kit are supplied in a container, such as a compartmentalized plastic enclosure. In some embodiments, the container has a hermetically sealable cover so that the contents of the kit can be sterilized and sealed for storage prior to use.

### Methods for Substantially Stable Storage of Blood Components

In another aspect of the present invention, methods for substantially stable storage of one or more polypeptide, nucleic acid molecule, or exosome in a blood sample in a native, non-denatured conformation or state are provided.

In certain embodiments, the methods comprise admixing a blood sample with a formulation for substantially stable storage of one or more polypeptide in a blood sample at ambient temperatures, wherein the one or more polypeptide is stabilized in a native, non-denatured state after storage at room temperature for a period of at least three days. In some embodiments, at least 80% of the polypeptides remain in a native, non-denatured state for a period of at least three days. In certain embodiments, the formulation for substantially stable storage of one or more non-denatured polypeptide in a blood sample at ambient temperatures comprises: a pH buffer; a non-reducing sugar; a trisaccharide; and at least one or more of a water-soluble polymer, a zwitterionic compound, a cationic compound or a phosphatase inhibitor. In certain embodiments, the formulation is one of the formulations set forth in Table 2. In some embodiments of the method, the one or more polypeptide remains in a native, non-denatured conformation after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

In some embodiments, the methods comprise admixing a blood sample with formulations for substantially stable storage of one or more nucleic acid molecule in a blood sample at ambient temperatures, wherein the one or more nucleic acid molecule is stabilized in a native, non-denatured state substantially free of contaminating intracellular nucleic acids after storage at room temperature for a period of at least three days. In some embodiments, at least 80% of the nucleic acids remain in a native, non-denatured state for a period of at least three days. In certain embodiments, the formulation for substantially stable storage of one or more non-denatured nucleic acid molecule in a blood sample at ambient temperature comprises: a pH buffer: a phosphatase inhibitor: a purine; a zwitterionic compound, a cationic compound, or a combination thereof; and an apoptosis inhibitor or a caspase inhibitor. In certain embodiments, the formulation comprises: a pH buffer: a phosphatase inhibitor; a purine: a zwitterionic compound or cationic compound; and a nuclease inhibitor. In some embodiments, the formulation further comprises: a polar solvent and a chelating agent. In some embodiments, the formulation further comprises dimethylacetamide and tripotassium EDTA. In certain embodiments, the formulation is one of the formulations set forth in Table 3. In some embodiments of the method, the one or more nucleic acid molecule remains in a native, non-denatured conformation after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

In some embodiments, the methods comprise admixing a blood sample with formulations for substantially stable storage of one or more exosome in a blood sample at ambient temperatures, wherein the one or more exosome is stabilized for a period of at least three days at ambient temperature. In some embodiments, at least 80% of the exosomes are stabilized for a period of at least three days. In certain embodiments, the formulation for substantially stable storage of one or more exosome in a blood sample at ambient temperature comprises: a pH buffer: a phosphatase inhibitor; a purine: a zwitterionic compound, a cationic compound, or a combination thereof; and an apoptosis inhibitor or a caspase inhibitor. In certain embodiments, the formulation comprises: a pH buffer; a phosphatase inhibitor; a purine; a zwitterionic compound or cationic compound; and a nuclease inhibitor. In some embodiments, the formulation further comprises: a polar solvent and a chelating agent. In some embodiments, the formulation further comprises dimethylacetamide and tripotassium EDTA. In certain embodiments, the formulation is one of the formulations set forth in Table 3. In some embodiments of the method, the one or more exosome is stabilized after storage at room temperature for a period of at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, or at least 18 days.

Blood collection tubes, bags, containers and vessels are well-known in the art and have been employed by medical practitioners for decades. Blood may be collected using any method or apparatus commonly employed by those skilled in the art such as venipuncture or finger prick. When the blood is collected by venipuncture, the formulations of the invention may be located inside the blood collection tube, e.g., an evacuated tube (VACUTAINER blood collection tube, Becton Dickenson or VACUETTE, Greiner) at the time that the blood sample is obtained from the subject, or the formulations may be added to an already obtained whole blood sample, preferably immediately or shortly after it is withdrawn.

The methods as described herein may use the articles of manufacture and kits disclosed herein.

The following Examples are presented by way of illustration and not limitation.

### Example 1

### Stabilization of Functionally Active Polypeptides in a Human Blood Sample for a Period of at Least Nine Days at Ambient Temperatures

This Example demonstrated formulations of the present invention, including those set forth for preparing substantially stable storage of native, functionally active polypeptides in a native, non-denatured conformation in a blood sample for a period of at least nine days at ambient temperatures.

The ability of the exemplary formulations A-G set forth in Table 2 to stabilize a plurality of polypeptides, e.g., a survey of 29 cytokines and chemokines, at ambient temperatures was examined using Millipex MAP Human cytokine/chemokine panel HCYTOMAG-60K-PX29 kit from Luminex Corp in accordance with the manufacturer's instructions.

Briefly, human whole blood samples were collected from donors and the blood was fractionated and the plasma layer was analyzed. A 75 or 135 (9:1 samples in Table 2) microliter aliquot of plasma was added to a 1.7 mL microfuge tube to which a solution of 29 different cytokines was added to spike the samples with an internal standard of 400 pg/mL, and the volume adjusted to 150 microliter. Control samples containing the internal standard in the absence (stored at −80° C.) or presence of plasma (fresh on assay day) were prepared and processed simultaneously with test samples. In addition, a standard curve was prepared using 16, 80, 400, 2,000, and 10,000 pg/mL, to ensure the linearity of the assay and to assist in proper quantitation of bound cytokine and chemokine concentrations.

Control and test samples were placed at room temperature and 25 microliter aliquots were removed at Day 0, Day 3 and Day 6. Day 0 and Day 3 samples were stored at −80° C. until processed on Day 6. To each 25 mL aliquot, 25 microliters each of Assay Buffer was added followed by the addition of 25 microliter amount of a human cytokine/chemokine antibody-immobilized magnetic beads coated with one of 29 antibodies directed against each member of the cytokine/chemokine panel was added. The antibodies recognize only non-denatured cytokine and chemokine polypeptides and denatured polypeptides will not bind to the beads. The mixtures were incubated overnight at 4° C. with shaking. The 96-well plate was placed against a magnet to sequester the bound magnetic beads, the supernatant was removed and the beads were washed twice with wash buffer.

To the washed beads, a 25 microliter aliquot of a Detection Antibody solution was added to each sample and incubated for one hour at room temperature. A 25 microliter aliquot of a streptavidin-phycoerythrin solution was added and the samples were stored at room temperature for 30 min. A magnet was placed next to the 96-well plate to sequester the beads, the supernatants were removed, and the samples were washed twice and resuspended in 150 microliters of wash buffer.

The amount of bound cytokine/chemokine from each plasma sample was determined using the Luminex Bio-Tek ELx405 instrument according to the manufacturer's instruction and the amount of each of the 29 members of the non-denatured cytokines and chemokines for each sample was calculated. The results for a subset of cytokines are shown in Table 14.

TABLE 4
Stabilization of Panel of Cytokines and Chemokines from a Blood
Sample at Ambient Temperatures Using the Formulations of Table 2.

| Cytokine | Formulation | Day 0 | Day 3 | Day 6 | Day 14 |
|---|---|---|---|---|---|
| IFNγ | NP | 100 | 78.10948 | 81.63109 | 19.41903 |
| | F | 100 | 106.5608 | 102.5185 | 85.24264 |
| IL-13 | NP | 100 | 80.85405 | 79.72953 | 45.60758 |
| | A | 100 | 105.8785 | 95.30526 | 70.70471 |
| | D | 100 | 89.69889 | 102.5402 | 77.19855 |
| | E | 100 | 105.8785 | 95.30526 | 70.70471 |
| | G | 100 | 89.69889 | 102.5402 | 77.19855 |
| TNFα | NP | 100 | 67.11964 | 51.49416 | 19.58567 |
| | A | 100 | 104.9812 | 80.6713 | 52.96681 |
| | G | 100 | 82.17989 | 69.07483 | 52.29539 |

NP: Non-protected control

### Example 2

### Stabilization of Circulating Free Nucleic Acids in Human Blood Sample for a Period of at Least Eight Days at Ambient Temperatures

This Example demonstrated formulations of the present invention, including those set forth Table 3, for preparing substantially stable storage of nucleic acid molecules in a native, non-denatured state in a blood sample while preventing substantial contamination from intracellular nucleic acids from lysed blood cells for a period of at least eight days at ambient temperatures.

The ability of the exemplary Formulations 1-10 set forth in Table 3 to stabilize a plurality circulating-free nucleic acids that are substantially free of contaminating intracellular nucleic acids in a blood sample was examined in two separate experiments. Briefly, 1.0 mL of the formulation was placed into three 5 mL Eppendorf tubes for each formulation, respectively. The non-protected tubes at room temperature and 4° C. received no formulation. Fresh human whole blood was collected from a healthy donor. The blood was pooled, mixed and then 4.0 mL aliquoted into each tube. After 10 tubes were loaded with blood the tubes were closed and mixed by inversion 10 times before adding blood to more tubes. The blood was remixed before addition to the next set of tubes. Once all the tubes were loaded and mixed the time zero tubes were spun at 3000 rpm for 10 minutes. Two 1.25 mL aliquots of the plasma were removed without disturbing the leukocyte and red blood cells, transferred to individual 1.5 mL Eppendorf microfuge tubes and spun at 16,000 g for 10 min at 4° C. The NP room temperature time zero blood was prepared by addition of 1 mL of 1×PBS pH 7.4. The tube was mixed by inversion 10 times and then the plasma prepared as described above. The two 1 mL cell-free plasma aliquots for each formulation tested were extracted using the MagNA Pure Compact Nucleic Acid Isolation Kit 1, Large Volume (Roche Cat #03 730 972 001) on the Roche MagNA Pure Compact Instrument from Roche Molecular Systems. The nucleic acids were eluted in 100 μL of elution buffer. The time zero samples were held at 4° C. until the day 4 samples were prepared as described above. The DNA extracted from each sample was analyzed by qPCR on a Bio-Rad CFX96 Real Time System (C1000 Touch Thermal Cycler) using a set of 18s rRNA primers from Qiagen (Cat #PPH05666E-200) and the Bio-Rad iQ SYBR Green Supermix (Cat #170-8882), 5 μL of each sample was used and the following plot generated. In addition, a 1:10 dilution of the isolated DNA was prepared and 5 μL of the dilution was also amplified by qPCR.

TABLE 5
Stabilization Circulating-Free Nucleic Acids Substantially
Free of Contaminating Intracellular Nucleic Acids
in a Blood Sample at Ambient Temperatures Using
the Formulations of Table 3. Yield (μg/mL)

| Day | 4° C. | RT | Form 1 | Form 2 | Form 3 | Form 4 | Form 5 |
|---|---|---|---|---|---|---|---|
| 0 | 0.02 | 0.02 | 0.02 | 0.02 | 0.02 | 0.02 | 0.02 |
| 4 | 0.17 | 0.19 | 0.14 | 0.04 | 0.02 | 0.07 | 0.07 |
| 8 | 0.06 | 0.91 | 0.10 | 0.06 | 0.08 | 0.03 | 0.08 |

TABLE 6
Stabilization Circulating-Free Nucleic Acids Substantially
Free of Contaminating Intracellular Nucleic Acids
in a Blood Sample at Ambient Temperatures Using
the Formulations of Table 3. Yield (μg/mL)

| Day | 4° C. | RT | Form 6 | Form 7 | Form 8 | Form 9 | Form 10 |
|---|---|---|---|---|---|---|---|
| 0 | 0.03 | 0.03 | 0.03 | 0.03 | 0.03 | 0.03 | 0.03 |
| 4 | 0.29 | 0.44 | 0.42 | 0.43 | 0.34 | 0.43 | 0.08 |
| 8 | 3.01 | 4.21 | 0.77 | 0.65 | 0.76 | 0.69 | 0.11 |

As shown in Tables 5 and 6, the addition of the exemplary formulations of Table 3 to human blood resulted in recovery of circulating-free nucleic acids that remained substantially free of contaminating intracellular DNA. At Day 0, the amount of circulating-free nucleic acids present in the test samples was essentially identical to controls and to other test samples; however, at Day 8, the blood stored at 4° C. or room temperature showed a significant increase the level of circulating-free nucleic acids shown by the increased yield in these samples from contaminating intracellular nucleic acids from lysed blood cells. In contrast, formulations 1-10 maintained the circulating-free nucleic acids substantially free of intracellular nucleic acids showing only modest increases at Day 8 and all of these formulations performed about as well or better than unprotected samples stored for 8 days at 4° C.

Unless the context requires otherwise, throughout the present specification and claims, the word “comprise” and variations thereof, such as, “comprises” and “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

Reference throughout this specification to “one embodiment” or “an embodiment” or “an aspect” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1-68. (canceled)

69. A composition for stabilizing one or more polypeptides in a native, non-denatured conformation in a blood sample at ambient temperatures, the composition comprising:
(i) a pH buffer;
(ii) at least one non-reducing sugar;
(iii) at least one trisaccharide; and
(iv) at least one of a phosphatase inhibitor and a water-soluble polymer.

70. The composition of claim 69, wherein the pH buffer comprises tris(hydroxymethyl) aminomethane (Tris) or phosphate-buffered saline (PBS).

71. The composition of claim 69, wherein the at least one non-reducing sugar is selected from the group consisting of sucrose and trehalose, or a combination thereof.

72. The composition of claim 69, wherein the at least one trisaccharide is selected from the group consisting of maltotriose, isomaltotriose, raffinose, melezitose, and nigerotriose, or any combination thereof.

73. The composition of claim 69, wherein the at least one phosphatase inhibitor is a serine-threonine phosphatase inhibitor.

74. The composition of claim 69, wherein the at least one phosphatase inhibitor is 2-glycerol phosphate.

75. The composition of claim 69, wherein the at least one water-soluble polymer is polyvinyl alcohol.

76. The composition of claim 69, wherein the at least one water-soluble polymer is polyvinyl alcohol having a molecular weight ranging from about 30-70,000 daltons.

77. The composition of claim 69, wherein the at least one water-soluble polymer is polyvinyl alcohol that is at least 80% hydrolyzed.

78. The composition of claim 69, further comprising one or more of a polyol, an amino acid, a non-ionic starch, a chelating agent, or a combination thereof.

79. The composition of claim 78, wherein the at least one amino acid is selected from the group consisting of glycine and sarcosine, or a combination thereof.

80. The composition of claim 78, wherein the polyol is selected from the group consisting of glycol, glycerol, erythritol, threitol, arabitol, xylitol, ribitol, adonitol, mannitol, sorbitol, galactitol, fucitol, iditol and inositol, or any combination thereof.

81. The composition of claim 78, wherein the non-ionic starch is hydroxyethyl starch (HES).

82. The composition of claim 78, wherein the chelating agent is selected from the group consisting of diethylenetriaminepentaacetic acid (DTPA), ethylenediaminetetraacetic acid (EDTA), ethylene glycol tetraacetic acid (EGTA), trans-1,2-diaminocyclohexane-N,N,N′,N′-tetraacetic acid (CDTA), 1,2-bis(2-aminophenoxy)ethane-N,N,N′,N′-tetraacetic acid (BAPTA), 1,4,7,10-tetraazacyclododecane-1,4,7,10-tetraacetic acid (DOTA), N-(2-hydroxyethyl)ethylenediamine-N,N′,N′-triacetic acid, sodium gluconate, and nitrilotriacetic acid (NTA).

83. A blood collection tube comprising the composition of claim 69.

84. The blood collection tube of claim 83, wherein the blood collection tube is an evacuated blood collection tube.

85. The blood collection tube of claim 83, further comprising a blood sample, wherein the blood sample is whole blood or plasma.

86. The blood collection tube of claim 85, wherein the ratio of the blood sample to the composition is about 9:1.

87. The blood collection tube of claim 85, wherein one or more polypeptides in the blood sample are stabilized in a native, non-denatured conformation after storage in the blood collection tube at ambient temperatures for a period of at least three days.

88. The blood collection tube of claim 87, wherein the sample is substantially free of contaminating intracellular polypeptides.

89. A kit comprising the blood collection tube of claim 83, and a package insert.

90. A method for stabilizing one or more polypeptides in a blood or plasma sample in a native, non-denatured conformation at ambient temperatures, the method comprising:
admixing a blood or plasma sample from a subject with a composition comprising:
(i) a pH buffer;
(ii) at least one non-reducing sugar;
(iii) at least one trisaccharide; and
(iv) at least one of a phosphatase inhibitor and a water-soluble polymer;
wherein one or more polypeptides are stabilized in a native, non-denatured conformation after storage at ambient temperatures for a period of at least three days.

91. The method of claim 90, wherein at least 80% of the one or more polypeptides remain in a native, non-denatured conformation after storage at room temperature for a period of at least three days.

92. The method of claim 90, wherein the composition further comprises one or more of a polyol, an amino acid, a non-ionic starch, a chelating agent, or a combination thereof.
